(19) United States
(12) Patent Application Publication
(10) Pub. No.: US 2004/0141928 A1
(43) Pub. Date: Jul. 22, 2004

(54) Composition

(75) Inventors: Melanie Gayle Brading, Merseyside (GB); Victoria Cromwell, Merseyside (GB); Steven Paul Rannard, Merseyside (GB); David William Thornthwaite, Merseyside (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/719,136

(22) Filed: Nov. 21, 2003

(30) Foreign Application Priority Data
Nov. 22, 2002 (EP) 02258069.0
Feb. 21, 2003 (EP) 03251046.3

(51) Int. Cl. A61K 7/16

(52) U.S. Cl. 424/49

(57) ABSTRACT

An oral care composition comprising a compound of Formula (1)

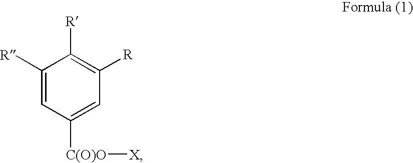

wherein R is either OH or Cl and R′ is either H or OH, wherein X is either a substituted or unsubstituted, straight chain or branched alkyl group having from 2 to 16 carbon atoms.

### BACKGROUND OF THE INVENTION

1. Field of the Invention

The present invention relates to an oral composition comprising a novel antimicrobial agent.

2. Related Art

Alkyl hydroxybenzoates (parabens) are known in the art where the alkyl group is methyl. For example, methyl hydroxybenzoate is mentioned, albeit fleetingly, for use in medicinal and oral care preparations as a preservative (WO 00/09507 and WO 00/69401).

### DESCRIPTION OF THE INVENTION

According to a first aspect the invention provides an oral care composition comprising a compound of Formula (1)

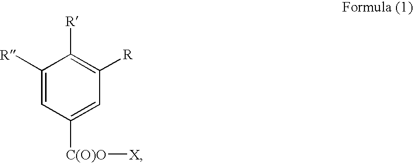

wherein R is either OH or Cl, R′ is either H or OH and R″ is either H or OH, wherein X is either a substituted or unsubstituted, straight chain or branched alkyl group having from 2 to 16 carbon atoms.

In a preferred embodiment where R is Cl it is preferred that R′ is OH and R″ is H.

Preferably the alkyl group is an aliphatic alkyl group, more preferably comprising from 1 to 16 and especially from 3 to 12 carbon atoms. Examples of suitable alkyl groups include methyl, ethyl, propyl, isopropyl, butyl, pentyl, hexyl, benzyl, heptyl, octyl, 2-ethyl hexyl, nonyl, decyl, undecyl, dodecyl or tridecyl. Of these the most preferred are the straight chain alkyls. The most preferred compound is where the alkyl group is n-octyl.

In the most preferred case the compound of Formula (1) is 3-hydroxybenzoic acid octyl ester; 3-chloro, 4-hydroxybenzoic acid octyl ester or 3,4-dihydroxybenzoic acid octyl ester. Most preferably, it is one of or a mixture of 3-hydroxybenzoic acid octyl ester and 3-chloro, 4-hydroxybenzoic acid octyl ester.

According to a second aspect the invention provides an oral care composition comprising a compound of Formula (2)

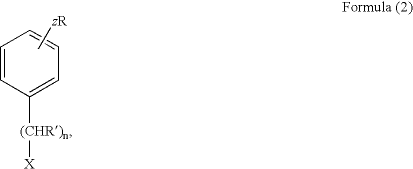

wherein:

R is a group independently selected from the group consisting of: H, F, Cl, Br, —OH, C₁₋₅ alkyl, —C(O)H, —C(O)C₁₋₅ alkyl, —OCH₃, —C₂H₅, —NH₂, —NHC(O)CH₃ and C(O)OC₁₋₆alkyl and z is from 1 to 5;

R′ is selected from the group consisting of: H, —OH, F, Cl, Br, I, and C₁-C₆ alkyl and n is an integer of from 0 to 12;

wherein X is —C(O)—R″ and R″ is —C₁₋₁₆ alkyl or —CH₂C₆H₆.

In a preferred embodiment R″ is a substituted or unsubstituted branched or straight chain hydrocarbon moiety comprising from 1 to 16 and especially from 5 to 10 carbon atoms. Examples of suitable R″ groups include pentyl, hexyl, benzyl, heptyl, octyl, 2-ethyl hexyl, nonyl, decyl, undecyl, dodecyl and tridecyl. Of these the most preferred are the straight chain alkyls. The most preferred active is where R″ is n-octyl.

According to Formula (2) z is from 1 to 5 and can be any number in between. Preferably z is 1.

According to Formula (2) R′ is selected from the group consisting of: H, —OH, F, Cl, Br, I, and C₁-C₆ alkyl. Preferably R′ is OH.

According to Formula (2) n is an integer of from 0 to 12. Preferably n is zero.

Preferably, at least one R group is in the para position.

Preferably, R is OH, more preferably, in the para position.

In the most preferred case the compound of Formula (2) is 1-(4-hydroxyphenyl)nonan-1-one.

According to a third aspect the invention provides an oral care composition comprising a compound of Formula (3)

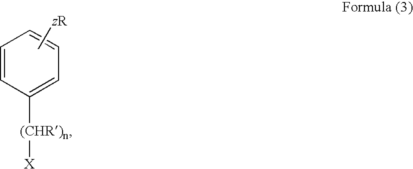

wherein:

R is a group independently selected from the group consisting of: H, F, Cl, Br, —OH, C₁₋₅ alkyl, —C(O)H, —C(O)C₁₋₅ alkyl, —OCH₃, —C₂H₅, —NH₂, —NHC(O)CH₃ and C(O)OC₁₋₆alkyl and z is from 1 to 5;

R′ is selected from the group consisting of: H, —OH, F, Cl, Br, I, and C₁-C₆ alkyl and n is an integer of from 0 to 12;

wherein X is —SO₂NH—R″ and R″ is —C-₁₆ alkyl or —CH₂C₆H₆.

In a preferred embodiment R″ is a substituted or unsubstituted branched or straight chain hydrocarbon moiety comprising from 1 to 16 and especially from 5 to 10 carbon atoms. Examples of suitable R″ groups include pentyl, hexyl, benzyl, heptyl, octyl, 2-ethyl hexyl, nonyl, decyl, undecyl, dodecyl and tridecyl. Of these the most preferred are the straight chain alkyls. The most preferred active is where R″ is n-octyl.

According to Formula (3) z is from 1 to 5, preferably 3. Preferably R is Cl or OH. More preferably and where z is 3, there are two R groups as Cl and one R group as OH. In this embodiment it is preferred that the two Cl groups are in positions 3 and 5 while the OH group is in position 6.

Preferably, n is zero.

Most preferably, the compound of Formula (3) is 3,5-dichloro, 2-hydroxy, N-octylbenzene sulphonamide.

According to a fourth aspect the invention provides an oral care composition comprising a compound of Formula (4)

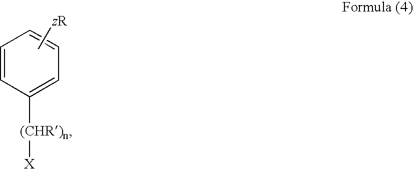

wherein:

R is a group independently selected from the group consisting of: —OH, C(O)OC₁₋₁₆ alkyl and z is from 1 to 5;

R′ is selected from the group consisting of: H, —OH, F, Cl, Br, I, and C₁-C₆ alkyl and n is an integer of from 0 to 12;

wherein X is —C(O)O—R″ and R″ is —C₁₋₁₆ alkyl or —CH₂C₆H₆.

In a preferred embodiment R or R″ is, independently from one another, a substituted or unsubstituted branched or straight chain hydrocarbon moiety comprising from 1 to 16 and especially from 1 to 8, more preferably 3 to 4 carbon atoms. Of these the most preferred are the straight chain alkyls. The most preferred active is where R″ is n-butyl. Preferably R and R″ are the same.

According to Formula (4) z is from 1 to 5 and can be any number in between. Preferably z is 2.

According to Formula (4) R′ is selected from the group consisting of: H, —OH, F, Cl, Br, I, and C₁-C₆ alkyl and n is an integer of from 0 to 12. Preferably n is zero.

The most preferred compound of Formula (4) is 5-hydroxy isophthalic acid dibutyl ester.

The compound according to Formulas 1 to 4 is preferably present at from about 0.1 to about 3.5% by weight of the composition. Preferably, in an amount ranging from about 0.15 to about 2.3% by weight and most preferably from about 0.2 to about 1.3% by weight of the composition.

The composition according to the invention may also comprise a halogenated hydroxydiphenyl ether compound, more preferably 2′,4,4′-trichloro-2-hydroxy-diphenyl ether, hereinafter known as triclosan. Preferably the halogenated hydroxydiphenyl ether is present at from 0.01 to 0.4% by weight of the composition.

The composition according to the invention may also comprise a divalent metal salt. Preferably, the divalent metal salt is a salt selected from the group consisting of zinc- and stannous salts such as zinc citrate, zinc sulphate, zinc glycinate, sodium zinc citrate, stannous pyrophosphate and mixtures thereof. The preferable divalent metal salt is zinc citrate.

Suitably, the amount of divalent metal salt ranges from about 0.01 to about 10% by weight of the composition, preferably from about 0.05 to about 5% by weight, more preferably from about 0.1 to about 2% by weight and especially preferably from about 0.3 to about 0.9% by weight of the composition.

The oral composition according to the invention comprise further ingredients which are common in the art, such as:

antimicrobial agents, e.g. chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition. Preferred abrasives are chalk and silica, more preferably fine ground natural chalk.

Humectants such as glycerol, sorbitol, propyleneglycol, xylitol, lactitol etc.;

binders and thickeners such as sodium carboxymethyl-cellulose, hydroxyethyl cellulose (Natrosol®), xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

In a fifth aspect the invention provides the use of a compound according to any of Formulas 1 to 4 in an oral care composition as an antimicrobial agent. Such use may be as an anti-tartar agent, anti-caries agent, anti-oral malodour agent, anti-gingivitis agent and any other related use for an antimicrobial agent in an oral composition.

In a sixth aspect the invention provides the use of a compound according to any of Formulas 1 to 4 in the manufacture of a medicament for the treatment or prevention of any one or more of gingivitis, oral malodour, tartar, tooth plaque build-up and caries.

In a seventh aspect the invention provides the use of a compound according to any of Formulas 1 to 4 as described herein as a delivery enhancing agent in an oral care composition for a halogenated diphenyl ether, preferably triclosan.

The description and examples illustrate selected embodiments of the present invention. In light thereof variations and modifications will be suggested to one skilled in the art, all of which are within the spirit and purview of this invention.

Embodiments according to the invention shall now be discussed with reference to the following non-limiting examples.

### EXAMPLE (1)

(1) 3-hydroxy benzoic acid octyl ester

(2) 4-Hydroxy-3-chlorbenzoic acid octyl ester [40664-24-2]

(3) Octyl-3,4-dihydroxybenzoate

(4) 4-Hydroxyphthalic acid dibutylester

(5) 1-(4-Hydroxyphenyl)nonan-1-one [14329-69-9]

(6) 3,5-Dichloro-2-hydroxy-N-octylbenzene sulphonamide

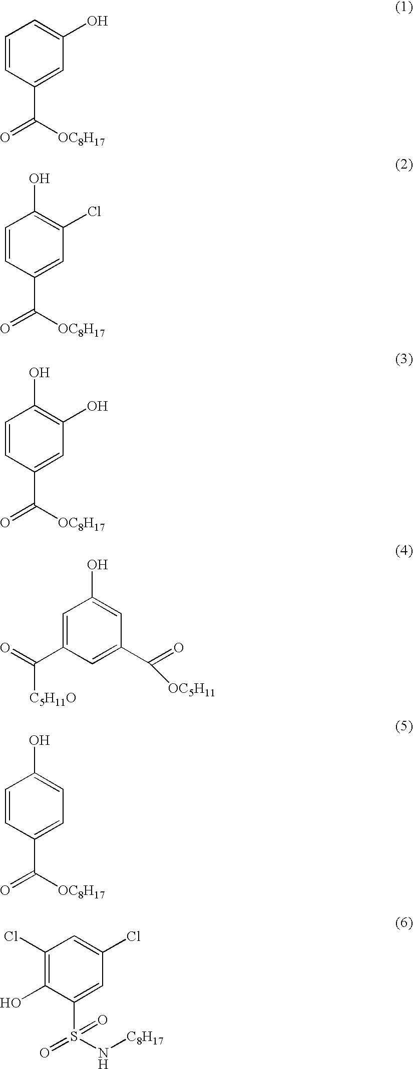

### EXAMPLE 2

For compounds 1-6 the synthesis starts from the parent acids, which are readily available, and these acids are just esterified using standard procedures.

Compound (5) is prepared from phenol via Friedel Crafts acylation.

For the sulphonamides the synthesis starts from the parent phenol/aniline, etc. by sulphonation with excess of chlorosulphonic acid to yield the sulphonyl chloride which is in turn further reacted with octyl amine to give the desired sulphonamide. (see example scheme)

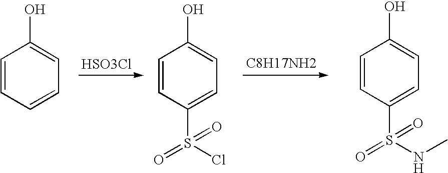

### EXAMPLE 3

The following is a formulation according to the present invention. It is made by known processes.

| Ingredient | % w/w |
|---|---|
| 70% aq. sorbitol | 45.0 |
| Saccharin | 0.2 |
| Polyethylene glycol | 2.0 |
| Titanium dioxide | 1.0 |
| Sodium fluoride | 0.32 |
| Thickening silica | 9.0 |
| Abrasive silica | 10.0 |
| SLS | 1.6 |
| Sodium carboxymethylcellulose | 0.8 |
| Flavour | 1.0 |
| Zinc citrate trihydrate | 0.75 |
| 3-hydroxybenzoic acid octyl ester | 1.0 |
| Water | to 100. |

1. An oral care composition comprising a compound of Formula (1) 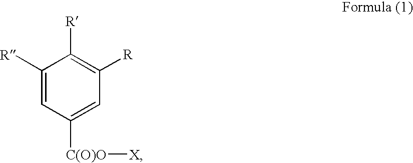
wherein R is either OH or Cl, R′ is either H or OH and R″ is either OH or H, wherein X is either a substituted or unsubstituted, straight chain or branched alkyl group having from 2 to 16 carbon atoms.

2. Composition according to claim 1, wherein in Formula (1) R is Cl, R′ is OH and R″ is H.

3. Composition according to claim 1 wherein the alkyl group has from 5 to 12 carbon atoms, more preferably 6 to 10 and most preferably 8 carbon atoms.

4. Composition according to claims 1, wherein the alkyl group is straight chain, unsubstituted octyl group.

5. Oral care composition comprising a compound of Formula (2) 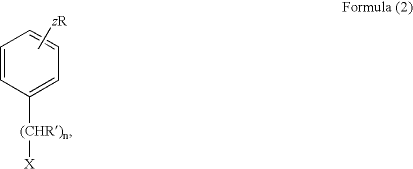
wherein:
R is a group independently selected from the group consisting of: H, F, Cl, Br, —OH, C₁₋₅ alkyl, —C(O)H, —C(O)C₁₋₅ alkyl, —OCH₃, —C₂H₅, —NH₂, —NHC(O)CH₃ and C(O)OC₁₋₆ alkyl and z is from 1 to 5;
R′ is selected from the group consisting of: H, —OH, F, Cl, Br, I, and C₁-C₆ alkyl and n is an integer of from 0 to 12;
wherein X is —C(O)—R″ and R″ is —C₁-₁₆ alkyl or —CH₂C₆H₆.

6. Oral care composition according to claim 5, wherein R″ is a substituted or unsubstituted branched or straight chain hydrocarbon moiety comprising from 1 to 16 and especially from 5 to 10 carbon atoms.

7. Composition according to claim 5, wherein R″ is n-octyl.

8. Composition according to claim 5, wherein R′ is independently selected from the group consisting of: H, —OH, F, Cl, Br, I, and C₁-C₆ alkyl.

9. Composition according to claim 5, wherein R′ is OH.

10. Oral care composition comprising a compound of Formula (3) 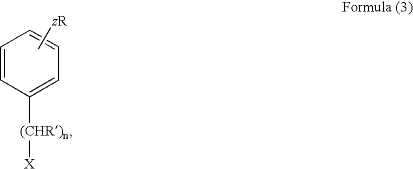
wherein:
R is a group independently selected from the group consisting of: H, F, Cl, Br, —OH, C₁₋₅ alkyl, —C(O)H, —C(O)C₁₋₅ alkyl, —OCH₃, —C₂H₅, —NH₂, —NHC(O)CH₃ and C(O)OC₁₋₆ alkyl and z is from 1 to 5;
R′ is selected from the group consisting of: H, —OH, F, Cl, Br, I, and C₁-C₆ alkyl and n is an integer of from 0 to 12;
wherein X is —SO₂NH—R″ and R″ is —C₁₋₁₆ alkyl or —CH₂C₆H₆.

11. Oral care composition according to claim 10, wherein R″ is a substituted or unsubstituted branched or straight chain hydrocarbon moiety comprising from 1 to 16 and especially from 5 to 10 carbon atoms.

12. Oral care composition according to claim 10, wherein R″ is a straight chain alkyl.

13. Oral care composition according to claim 10 where R″ is n-octyl.

14. Oral care composition according to claim 10, wherein R is Cl or OH.

15. Oral care composition according to claim 10, wherein there are two R groups as Cl and one R group as OH.

16. Oral care composition according to claim 15, wherein the two Cl groups are in positions 3 and 5 while the OH group is in position 6.

17. Oral care composition comprising a compound of Formula (4) 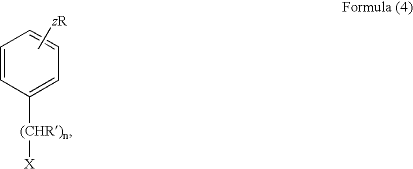
wherein:
R is a group independently selected from the group consisting of: —OH, C(O)OC₁₋₁₆ alkyl and z is from 1 to 5;
R′ is selected from the group consisting of: H, —OH, F, Cl, Br, I, and C₁-C₆ alkyl and n is an integer of from 0 to 12;
wherein X is —C(O)O—R″ and R″ is −C₁₋₆ alkyl or —CH₂C₆H₆.

18. Oral care composition according to claim 17, wherein R″ is a substituted or unsubstituted branched or straight chain hydrocarbon moiety comprising from 1 to 16 and especially from 1 to 8, more preferably 3 to 4 carbon atoms.

19. Oral care composition according to claim 17, wherein R″ is n-butyl.
